SEQUENCE LISTING (14 sequences)

SEQ ID NO: 1 - protein, 11 aa, synthetic construct
  REGION 1..11: note = Synthetic peptide
RRRRRRRRRRR

SEQ ID NO: 2 - skipped

SEQ ID NO: 3 - protein, 6 aa, synthetic construct
  REGION 1..6: note = Synthetic Peptide
AAAAAK

SEQ ID NO: 4 - protein, 7 aa, synthetic construct
  REGION 1..7: note = Synthetic Peptide
AAAAAAK

SEQ ID NO: 5 - protein, 8 aa, synthetic construct
  REGION 1..8: note = Synthetic Peptide
AAAAAAAK

SEQ ID NO: 6 - protein, 7 aa, synthetic construct
  REGION 1..7: note = Synthetic Peptide
AAAAAKK

SEQ ID NO: 7 - protein, 8 aa, synthetic construct
  REGION 1..8: note = Synthetic Peptide
AAAAAAKK

SEQ ID NO: 8 - protein, 9 aa, synthetic construct
  REGION 1..9: note = Synthetic Peptide
AAAAAAAKK

SEQ ID NO: 9 - protein, 6 aa, synthetic construct
  REGION 1..6: note = Synthetic Peptide
AAAAAR

SEQ ID NO: 10 - protein, 7 aa, synthetic construct
  REGION 1..7: note = Synthetic Peptide
AAAAAAR

SEQ ID NO: 11 - protein, 8 aa, synthetic construct
  REGION 1..8: note = Synthetic Peptide
AAAAAAAR

SEQ ID NO: 12 - protein, 7 aa, synthetic construct
  REGION 1..7: note = Synthetic Peptide
AAAAARR

SEQ ID NO: 13 - protein, 8 aa, synthetic construct
  REGION 1..8: note = Synthetic Peptide
AAAAAARR

SEQ ID NO: 14 - protein, 9 aa, synthetic construct
  REGION 1..9: note = Synthetic Peptide
AAAAAAARR

(19) United States
(12) Patent Application Publication
(10) Pub. No.: US 2025/0020668 A1
(43) Pub. Date: Jan. 16, 2025

(54) CANCER LESION TISSUE EVALUATION FOR OPTIMIZING EFFECT OF BORON NEUTRON CAPTURE THERAPY

(71) Applicant: 3-D Matrix, Ltd., Cambridge, MA (US)

(72) Inventors: Hideki MATSUI, Okayama-shi (JP); Atsushi FUJIMURA, Okayama-shi (JP)

(73) Assignee: 3-D Matrix, Ltd., Tokyo (JP)

(21) Appl. No.: 18/814,981

(22) Filed: Aug. 26, 2024

Related U.S. Application Data
(62) Division of application No. 16/970,199, filed on Aug. 14, 2020, now abandoned.

(30) Foreign Application Priority Data
Feb. 15, 2018 (JP) 2018-025216

(51) Int. Cl. G01N 33/68; A61K 33/22; G01N 33/50

(52) U.S. Cl. CPC G01N 33/6893; A61K 33/22; G01N 33/5008

(57) ABSTRACT

The present invention relates to a method, a kit, etc., for predicting the response of a BNCT using a BSH-related medicine-containing boron preparation and a BPA-containing boron preparation, the method and kit being characterized by examining the expression of CD44, a translation-related factor and/or LAT1 in cancer cells of a sample. When the expression of the CD44 or translation-related factor in the cancer cells of the sample is high, it can be predicted that the BNCT using the BSH-related medicine-containing boron preparation is likely to be responsive. When the expression of the LAT1 in the cancer cells of the sample is high, it can be predicted that the BNCT using the BPA-containing boron preparation is likely to be responsive.

### CONTINUITY

This application is a divisional of U.S. application Ser. No. 16/970,199, filed Aug. 14, 2020, which is a National Stage entry of International Application No. PCT/JP2019/005727, filed Feb. 8, 2019, which claims priority to Japanese Application No. 2018-025216, filed Feb. 15, 2018. The entire contents of each of these applications are incorporated herein by reference in their entirety.

### SEQUENCE LISTING

The instant application contains a Sequence Listing which is being submitted electronically and is hereby incorporated by reference in its entirety. Said Sequence Listing, created on Aug. 23, 2024, is named 3DM-18-02-BORON_US02-SequenceListing.xml and is 17,445 bytes in size.

### BACKGROUND OF THE INVENTION

### Field of the Invention

The present invention relates to a method for predicting the effect of boron neutron capture therapy (BNCT) and a kit therefor.

### Background Art

BNCT is a method of cancer treatment by administering an agent containing boron (a boron agent) to a cancer patient, selectively incorporating a boron atom into the cancer cell, and irradiating neutrons to the affected area. In this method, a boron atom in the boron agent captures a neutron and splits into an alpha particle (a helium atomic nucleus) and a lithium atomic nucleus. Cancer cells are selectively destroyed by the energy of the alpha particle. Currently, p-boronophenylalanine (BPA) is mainly used in BNCT as a boron agent. However, there are many carcinomas that have not been found to yield sufficient results with BNCT using conventional BPA or that have not been considered to date a good indication for BNCT.

To predict the dosing effect of BPA, tissue aggregation of F-BPA has been evaluated by PET imaging, utilizing that F-BPA to which a radioisotope of fluorine is chemically bound is taken up in the same manner as BPA from amino acid transporters such as LAT1 (see, for example, Non Patent Literatures 1 to 5). However, because of the limited facilities available for PET imaging and the high price of the agents, it is not easy to predict the effects. In addition, no effective means have been found to evaluate the uptake into cells of boron agents other than BPA.

### CITATION LIST

### Non Patent Literature

Non Patent Literature 1: EJNMMI Res. 2014 Dec. 20; 4 (1): 70. doi: 10.1186/s13550-014-0070-2. eCollection 2014 December.
Non Patent Literature 2: J Nucl Med. 2004 February; 45 (2): 302-8.
Non Patent Literature 3: J Nucl Med. 2005 November; 46 (11): 1858-65.
Non Patent Literature 4: Ann Nucl Med. 2016 December; 30 (10): 749-755. Epub 2016 Sep. 1.
Non Patent Literature 5: Appl Radiat Isot. 2009 July; 67 (7-8 Suppl): S348-50. doi: 10.1016/j.apradiso.2009.03.061. Epub 2009 Mar. 27.

### SUMMARY OF THE INVENTION

### Problem to be Solved

It has been required to perform effective BNCT for carcinomas that have not been found to yield sufficient results by BNCT using conventional BPA or that have not been considered to date a good indication for BNCT. For that, methods are needed to easily predict the effects of boron agents that differ from BPA. It is also required to select the optimal boron formulation for each carcinoma/case.

### Means to Solve the Problem

In order to solve the above problem, the present inventors conducted intensive studies and have found that certain boron agents, a complex comprising a peptide containing a basic amino acid residue and mercaptoundecahydrodecaborate (BSH), and BSH to which a peptide containing a basic amino acid residue covalently linked, are taken into a cancer cell through a pathway via CD44, and that BSH, a complex comprising BSH, or a BSH derivative targets a translation system in the cell, leading to the completion of the present invention.

That is, the present invention provides the following.

(1) A method for predicting efficiency of introduction into a cancer cell of a complex comprising a peptide containing a basic amino acid residue and BSH, or BSH to which a peptide containing a basic amino acid residue is covalently linked, the method comprising examining expression of CD44 in the cancer cell in a sample.

(2) The method according to (1), wherein the peptide containing a basic amino acid residue contains an arginine residue and/or a lysine residue and is 3 to 13 amino acids in length.

(3) The method according to (1) or (2), wherein the expression of CD44 is examined by immunostaining.

(4) A kit for predicting efficiency of introduction into a cancer cell of a complex comprising a peptide containing a basic amino acid residue and BSH, or BSH to which a peptide containing a basic amino acid residue is covalently linked, the kit comprising means for examining expression of CD44.

(5) A method for predicting a sensitivity of a cancer cell to a boron neutron capture therapy (BNCT) using a complex comprising a peptide containing a basic amino acid residue and BSH, or BSH to which a peptide containing a basic amino acid residue is covalently linked, the method comprising examining expression of CD44 in the cancer cell in a sample.

(6) The method according to (5), wherein the peptide containing a basic amino acid residue contains an arginine residue and/or a lysine residue and is 2 to 20 amino acids in length.

(7) The method according to (5) or (6), wherein the expression of CD44 is examined in immunostaining.

(8) A kit for determining a sensitivity of a cancer cell to a BNCT using a complex comprising a peptide containing a basic amino acid residue and BSH, or BSH to which a peptide containing a basic amino acid residue is covalently linked, the kit comprising means for examining expression of an anti-CD44 antibody.

(9) A method for predicting a residence time of BSH, a complex comprising BSH, or a BSH derivative in a cancer cell, the method comprising examining expression of a translation-related factor in the cancer cell in a sample.

(10) The method according to (9), wherein the translation-related factor is one or more selected from the group consisting of eIF4A, eIF4E, eIF4G, eEF2, eRF3, pS6, and PABPc1.

(11) The method according to (9) or (10), wherein the BSH derivative is BSH to which a peptide containing a basic amino acid residue is covalently linked.

(12) The method according to (11), wherein the peptide containing a basic amino acid residue contains an arginine residue and/or a lysine residue and is 2 to 20 amino acids in length.

(13) The method according to any one of (9) to (12), wherein the expression of a translation-related factor is examined by immunostaining.

(14) A kit for predicting a residence time of BSH, a complex comprising BSH, or a BSH derivative in a cancer cell, the kit comprising means for examining expression of a translation-related factor.

(15) A method for predicting a sensitivity of a cancer cell to BNCT using BSH, a complex comprising BSH, or a BSH derivative, the method comprising examining expression of a translation-related factor in the cancer cell in a sample.

(16) The method according to (14), wherein the translation-related factor is one or more selected from the group consisting of eIF4A, eIF4E, eIF4G, eEF2, eRF3, pS6, and PABPc1.

(17) The method according to (15) or (16), wherein the BSH derivative is BSH to which a peptide containing a basic amino acid residue is covalently linked.

(18) The method according to (17), wherein the peptide containing a basic amino acid residue contains an arginine residue and/or a lysine residue and is 2 to 20 amino acids in length.

(19) The method according to any one of (15) to (18), wherein the expression of a translation-related factor is examined by immunostaining.

(20) A kit for determining a sensitivity of a cancer cell to BNCT using BSH, a complex comprising BSH, or BSH derivative, the kit comprising means for examining expression of a translation-related factor.

(21) A method for predicting a possibility that BNCT is effective using a complex comprising a peptide containing a basic amino acid residue and BSH, or BSH to which a peptide containing a basic amino acid residue is covalently linked, the method comprising: examining an expression of CD44 in a cancer cell in a sample; and predicting that the higher the expression of CD44, the higher the possibility that BNCT is effective using a complex comprising a peptide containing a basic amino acid residue and BSH, or BSH to which a peptide containing a basic amino acid residue is covalently linked.

(22) A kit for predicting a possibility that BNCT is effective using a complex comprising a peptide containing a basic amino acid residue and BSH, or BSH to which a peptide containing a basic amino acid residue is covalently linked, the kit comprising means for examining expression of CD44.

(23) A method for predicting a possibility that BNCT is effective using BSH, a complex comprising BSH, or a BSH derivative, the method comprising: examining an expression of a translation-related factor in a cancer cell in a sample; and predicting that the higher the expression of the translation-related factor, the higher the possibility that BNCT is effective using BSH, a complex comprising BSH, or a BSH derivative.

(24) A kit for predicting a possibility that BNCT is effective using BSH, a complex comprising BSH or a BSH derivative, the kit comprising means for examining expression of a translation-related factor.

(25) A method for selecting a boron formulation for BNCT, comprising the steps of:
(a) examining expression of CD44 and expression of LAT1 in a cancer cell in a sample; and then
(b) selecting a boron formulation containing a complex comprising a peptide containing a basic amino acid residue and BSH, or BSH to which a peptide containing a basic amino acid residue is covalently linked when the expression of CD44 is higher than the expression of LAT1, and selecting a boron formulation containing BPA when the expression of LAT1 is higher than the expression of CD44.

(26) A kit for use in selecting a boron formulation for BNCT, the kit comprising means for examining expression of CD44 and means for examining expression of LAT1.

(27) A method for selecting a boron formulation for BNCT, comprising the steps of:
(a) examining expression of a translation-related factor and expression of LAT1 in a cancer cell in a sample; and then
(b) selecting a boron formulation containing BSH, a complex comprising BSH, and/or a BSH derivative when the expression of a translation-related factor is higher than the expression of LAT1, and selecting a boron formulation containing BPA when the expression of LAT1 is higher than the expression of a translation-related factor.

(28) A kit for use in selecting a boron formulation for BNCT, the kit comprising means for examining expression of a translation-related factor and means for examining expression of LAT1.

### Effects of the Invention

According to the present invention, there are provided a method and a kit for predicting an effect of BNCT when a complex comprising a peptide containing a basic amino acid residue and BSH, or BSH to which a peptide containing a basic amino acid residue is covalently linked are used as a boron agent. As a result, it is possible to expand BNCT indication cases and improve BNCT response rates, by the use of a complex comprising a peptide containing a basic amino acid residue and BSH, and BSH to which a peptide containing a basic amino acid residue is covalently linked for carcinomas that have not been found to yield sufficient results with BNCT using conventional BPA or that have not been considered to date a good indication for BNCT. In addition, the method and kit of the present invention do not require special instruments or procedures, and can be easily used in the general hospitals and laboratories. Furthermore, according to the present invention, there is provided a method and a kit for selecting an optimal boron formulation for each carcinoma/case. That is, by examining the expression pattern of the initial target proteins of the various boron formulations, the optimal boron formulation combination and dosage can be determined for each carcinoma/case, and the BNCT response rate can be improved. Thus, according to the present invention, the potential of BNCT can be expanded, failures in finding cases can be reduced, and personalized medicine of BNCT can be achieved.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram showing uptakes of BSH-3R, BSH-11R, and BSH/A6K complex into CD44-expressing cancer cells and CD44-knocked down cancer cells. shControl represents a malignant brain tumor cell line in which the CD44 gene is not knocked down (control), and shCD44 #1 and shCD44 #2 represent malignant brain tumor cell sublines in which the CD44 gene is knocked down. The upper section is the results of confirming the knockdown of CD44. The middle section is fluorescent immunostaining images of the control and the CD44-knocked down cell sublines with BSH antibodies. The lower section is the breakdown (relative number) of BSH antibody staining intensities of the control and the CD44-knocked down cell sublines when BSH-11R was used.

FIG. 2 is an immunoprecipitation pattern showing that BSH-11R targets a translation system in cancer cells.

FIG. 3 is an immunoprecipitation pattern showing that BSH-11R targets a translation in cancer cells.

FIG. 4 is a graph showing the destruction of the translation system when cancer cells were pretreated with BSH-11R and subjected to BNCT. The solid line indicates logarithms of relative amounts of 18S ribosomal RNA, the broken line indicates logarithms of relative amounts of CD44 mRNA, and the dotted line indicates logarithms of relative amounts of TAZ mRNA.

FIG. 5 shows the results of examining LAT1 and CD44 expressions in various carcinoma specimens (the upper section in the figure). Expressions of LAT1 and CD44 were examined for each type of malignant brain tumors (the lower section in the figure). In FIG. 5, the higher the expression intensity, the more intense the red color, and the lower the expression intensity, the more intense the green color. When the expression intensity is moderate, it is indicated in black color. In the figure, one line shows the expression in one specimen.

FIG. 6 shows the results of examining expressions of LAT1 and CD44 for each type of breast cancer. In FIG. 6, the higher the expression intensity, the more intense the red color, and the lower the expression intensity, the more intense the green color. When the expression intensity is moderate, it is indicated in black color. In the figure, one line shows the expression in one specimen.

### DETAILED DESCRIPTION OF THE INVENTION

In the first aspect, the present invention provides a method for predicting efficiency of introduction into a cancer cell of a complex comprising a peptide containing a basic amino acid residue and BSH, or BSH to which a peptide containing a basic amino acid reside is covalently linked, the method comprising examining expression of CD44 in the cancer cell in a sample.

CD44 is a cell membrane protein involved in cell adhesion, cell movement, and proliferation, infiltration, and metastasis of cancer cells, and the like. The present inventors have found for the first time that CD44 is a target for introduction into cells of a complex comprising a peptide containing a basic amino acid residue and BSH, and BSH to which a peptide containing a basic amino acid is covalently linked. That is, it has been found that a complex comprising a peptide containing a basic amino acid residue and BSH, and BSH to which a peptide containing a basic amino acid residue is covalently linked are easily introduced into cancer cells with high expression of CD44. Thus, in the above method, it can be predicted that the higher the expression of CD44 in a cancer cell, the higher the efficiency of introduction into a cancer cell of a complex comprising a peptide containing a basic amino acid residue and BSH, or BSH to which a peptide containing a basic amino acid residue is covalently linked.

The sample may be any sample as long as it contains a cancer cell. For example, the sample may be a pathological specimen or a biopsy specimen. Alternatively, the sample may be a body fluid sample, such as blood.

The cancer may be any type of cancer. Examples include, but are note limited to, epithelial cancers including a breast cancer, a pancreatic cancer, and an oral cancer, a brain tumor, and refractory cancers such as a bone and soft tissue tumor.

In the method of this aspect, the expression amount of CD44 in a cancer cell is examined. As used herein, CD44 means either one or both of a CD44 gene and a CD44 protein. Expression of CD44 can be examined by known methods. For example, expression of CD44 can be examined by detecting a CD44 protein in a cancer cell by immunostaining with an anti-CD44 antibody, or by examining expression of a CD44 gene in a cancer cell by Northern blot analysis or real-time PCR. Means and methods for examining expression of CD44 in a cancer cell are not limited thereto.

Examples of preferred methods for examining expression of CD44 include immunostaining with an anti-CD44 antibody. Anti-CD44 antibodies can be obtained by know methods and are also commercially available. The anti-CD44 antibody may be a polyclonal antibody or a monoclonal antibody, and preferably, a monoclonal antibody. Immunostaining is a know method. It is preferable to use an anti-CD44 antibody to which a detectable label such as a fluorescent dye, a fluorescent protein, or a radioisotope is attached, for immunostaining. Secondary antibodies may also be used to detect the anti-CD44 antibody. The anti-CD44 antibody may be directed to a CD44 protein or a portion thereof, or may be directed to a gene encoding a CD44 protein or a portion thereof.

Northern blot analysis or real-time PCR is also preferably used to examine expression of CD44. These methods are known to those skilled in the art.

Determination of whether CD44 expression is high can also be performed by unknown methods. For example, whether the expression of CD44 is high can be determined using the expression of CD44 in a cell from a normal subject or in a cell from a patient with a benign disease such as a benign tumor as a basis. Whether the expression of CD44 is high may also be determined using the expression amount of a housekeeping gene such as glyceraldehyde-3-phosphate dehydrogenase (GAPDH) or β-actin or of a product thereof as a basis.

A complex comprising a peptide containing a basic amino acid residue and BSH can be obtained by mixing, and conjugating a peptide containing a basic amino acid residue to BSH in an aqueous solution. Optionally, the size of the complex may be adjusted using, for example, an extruder having an appropriate pore size. The method for producing the complex comprising a peptide containing a basic amino acid residue and BSH may be a similar method to that described in International Patent Application Publication WO 2018/097335 A1 which is incorporated herein by reference.

BSH to which a peptide containing a basic amino acid residue is covalently linked can be obtained by known methods. For example, a peptide containing a basic amino acid residue may be covalently linked to BSH via an SH group of BSH.

The peptide containing a basic amino acid residue contains at least one basic amino acid residue. An amino acid residue constituting the peptide containing a basic amino acid residue may be a natural amino acid residue or a non-natural amino acid residue, and may be an L-form or a D-form. The basic amino acid residue is known, and typical examples thereof include an arginine residue, a lysine residue, and a histidine residue. Other examples of the basic amino acid residue include, but are not limited to, an ornithine residue and a citrulline residue. Preferred basic amino acid residues are an arginine residue and a lysine residue. Thus, the preferred peptide containing a basic amino acid residue in the present invention is a peptide containing an arginine residue and/or a lysine residue. The length of the peptide containing a basic amino acid residue is not particularly limited, but generally 2 to 20 amino acids, preferably 3 to 16 amino acids, more preferably 4 to 14 amino acids, still more preferably 6 to 13 amino acids.

The peptide containing a basic amino acid residue, which forms a complex with BSH, is preferably a peptide containing a basic amino acid residue and a hydrophobic amino acid residue. The basic amino acid residue is as described above. The hydrophobic amino acid residue is also known, and examples thereof include, but are not limited to, a glycine residue, an alanine residue, a valine residue, a leucine residue, and isoleucine residue, a methionine residue, a proline residue, a phenylalanine residue, a tryptophan residue, a tyrosine residue, a methionine residue, and a cysteine residue.

The more preferred peptide containing a basic amino acid residue, which forms a complex BSH, is a peptide containing an arginine residue and/or a lysine residue. Examples of the peptide containing a basic amino acid residue, which forms a complex with BSH, include, but are not limited to, AAAAAK, AAAAAAK, AAAAAAAK, AAAAAKK, AAAAAAKK, AAAAAAAKK, AAAAAR, AAAAAAR, AAAAAAAR, AAAAARR, AAAAAARR, AAAAAAARR, in one letter amino acid notation. More preferred examples of the peptide containing a basic amino acid residue, which forms a complex with BSH include, but are not limited to, AAAAAAK and AAAAAAR.

The preferred peptide containing a basic amino acid residue, which is covalently linked to BSH, is a peptide containing an arginine residue and/or a lysine residue. It is preferred that the isoelectric point of the peptide containing a basic amino acid residue, which is covalently linked to BSH, is higher than 7. Alternatively, 50% or more, more preferably 60% or more, still preferably 70% or more, and still more preferably 80% or more of the amino acid residues constituting the peptide containing a basic amino acid residue, which is covalently linked to BSH, are basic amino acid residues. Preferred examples of the peptide containing such basic amino acid residues include, but are not limited to 3 to 16 mers of arginine and 3 to 16 mers of lysine. More preferred examples of the peptide containing a basic amino acid residue, which is covalently linked to BSH, include, but are not limited to, 4 to 14 mers or arginine or 4 to 14 mers of lysine, still preferably 6 to 13 mers of arginine or 6 to 13 mers of lysine.

In the second aspect, the present invention provides a kit for predicting efficiency of introduction into a cancer cell of a complex comprising a peptide containing a basic amino acid residue and BSH, or BSH to which a peptide containing a basic amino acid residue is covalently linked, the kit comprising means for examining expression of CD44. The kit can be used to perform the method of the first aspect. The means for examining expression of CD44 is known to those skilled in the art, and is not particularly limited, but preferred examples of the means include immunostaining with an anti-CD44 antibody, and expression analysis of a CD44 gene using Northern blot analysis or real-time PCR. The kit may thus comprise a reagent or an instrument for performing immunostaining with an anti-CD44 antibody. Alternatively, the kit may comprise a reagent or an instrument for Northern blot analysis or real-time PCR for examining expression of CD44. The kit typically comprises an instruction.

In the third aspect, the present invention provides a method for predicting a sensitivity of a cancer cell to a boron neutron capture therapy (BNCT) using a complex comprising a peptide containing a basic amino acid residue and BSH or BSH to which a peptide containing a basic amino acid residue is covalently linked, the method comprising examining expression of CD44 in the cancer cell in a sample.

Since the complex comprising a peptide containing a basic amino acid residue and BSH, and BSH to which a peptide containing a basic amino acid residue is covalently linked are easily introduced into cancer cells with high expression of CD44, cancer cells with high expression of CD44 are highly sensitive to BNCT using the complex comprising a peptide containing a basic amino acid residue and BSH and/or BSH to which a peptide containing a basic amino acid residue is covalently linked. Thus, in the above method, it can be predicted that the higher the expression of CD44 in a cancer cell, the higher the sensitivity of the cancer cell to BNCT using a complex comprising a peptide containing a basic amino acid residue and BSH, or BSH to which a peptide containing a basic amino acid residue is covalently linked.

In the fourth aspect, the present invention provides a kit for determining a sensitivity of a cancer cell to BNCT using a complex comprising a peptide containing a basic amino acid residue and BSH, or BSH to which a peptide containing a basic amino acid residue is covalently linked, the kit comprising means for examining expression of an anti-CD44 antibody. The kit can be used to perform the method of the third aspect. The kit typically comprises an instruction.

According to the method of the third aspect and the kit of the fourth aspect of the present invention, it is not only possible to know whether a cancer of a patient is a case suitable for BNCT using a complex comprising a peptide containing a basic amino acid residue and BSH and/or BSH to which a peptide containing a basic amino acid residue is covalently linked, but also to optimize the type of appropriate boron formulation in BNCT (for example, whether a complex comprising a peptide containing a basic amino acid residue and BSH and/or BSH to which a peptide containing a basic amino acid residue is covalently linked is effective, BPA is effective, or a combination thereof is effective) and to optimize the dosage or the compounding amount of the boron formulation.

For other details of the method of the third aspect and the kit of the fourth aspect of the present invention, the descriptions for the method of the first aspect and the kit of the second aspect of the present invention are applied.

In the fifth aspect, the present invention provides a method for predicting a residence time of BSH, a complex comprising BSH, or a BSH derivative in a cancer cell, the method comprising examining expression of a translation-related factor in the cancer cell in a sample.

The complex comprising BSH is one in which BSH and another substance form a complex in a manner other than a covalent bond, such as an ionic bond, a van der Waals force, or a hydrophobic bond. Examples of the complex comprising BSH include, but are not limited to, a complex comprising a peptide containing a basic amino acid residue and BSH, as described above. The BSH derivative is one in which another molecule is covalently linked to BSH via any atom of the BSH. Examples of the BSH derivative include, but are not limited to, one in which a peptide containing a basic amino acid residue, as described above, is covalently linked to BSH. Examples of the complex comprising BSH include, but are not limited to, a complex comprising a peptide containing a basic amino acid residue and BSH. Examples of the complex comprising a peptide containing a basic amino acid residue and BSH include, but are not limited to, BSH/A6K. Examples of the BSH derivative include, but are not limited to, BSH to which a peptide containing a basic amino acid residue is covalently linked. Examples of the BSH to which a peptide containing a basic amino acid residue is covalently linked include, but are not limited to, BSH-3R and BSH-11R. The details of the peptide containing a basic amino acid residue is as described above (see Example 1, for BSH/A6K, BSH-3R, BSH-11R).

The present inventors have found for the first time that BSH to which a peptide containing a basic amino acid residue is covalently linked targets translation-related factors in a cell. The high expression of a translation-related factor that is an intracellular target means that the translation-related factor in the cell is abundant. Accordingly, in a cancer cell with high expression of the translation-related factor, the residence time of BSH to which a peptide containing a basic amino acid residue is covalently linked is long. It can thus be predicted that the higher the expression of a translation-related factor in a cancer cell, the longer the residence time of BSH, a complex comprising BSH, or a BSH derivative in the cancer cell.

In the method of this aspect, the expression amount of a translation related factor in a cancer cell is examined. Translation-related factors are factors involved in the translation of genetic information in cells, and they are known. As used herein, the translation-related factor refers to either one or both of a gene encoding the translation-related factor and a product thereof. Expression of one kind or two or more kinds of the translation-related factors may be examined. Examples of the translation-related factor include, but are not limited to, translational initiation factors such as eIF4A (including eIF4A1), eIF4E, and eIF4G, translational elongation factors such as eEF2, translational termination factors such as eRF3, ribosomal proteins such as pS6, and mRNA binding factors such as PABPc1. In the method of this aspect, the expression of one kind of translation-related factor may be examined, but it is preferable that expression of two or more kinds of the translation-related factors may be examined.

Among the translation-related factors, eIF4A (including eIF4A1), eIF4E, eIF4G, eEF2, eRF3, pS6, and the like are targeted by a peptide containing a basic amino acid residue covalently linked to BSH. PA3Pc1 is targeted by any type of BSH formulations, including simple BSH.

Expression of a translation-related factor can be examined by known methods. For example, expression of a translation-related factor may be examined by detecting a translation-related factor in a cancer cell by immunostaining with an antibody against the translation-related factor, or by examining expression of a gene of a translation-related factor in a cancer cell by Northern blot analysis or real-time PCR. Means and methods for examining expression of a translation-related factor in a cancer cell are not limited thereto.

Preferably, expression of a translation-related factor is examined by immunostaining with an antibody against a translation-related factor. Antibodies against translation-related factors can be obtained by known methods and are also commercially available. The antibody against a translation-related factor may be a polyclonal antibody or a monoclonal antibody, and preferably, a monoclonal antibody. Immunostaining is a known method. It is preferable to use an antibody against a translation-related factor to which a detectable label such as fluorescent dye, a fluorescent protein, or a radioisotope is attached, for immunostaining. Secondary, antibodies may also be used to detect the antibody against a translation-related factor. The antibody against a translation-related factor may be an antibody against a translation related factor as a protein, or a portion thereof, or may be an antibody against a gene encoding a translation related factor as a protein, or a portion thereof.

It is also preferable to examine expression of translation-related factors by Northern blot analysis or real-time PCR. These methods are known to those skilled in the art.

Determination of whether expression of a translation-related factor is high can also be performed by known methods. For example, whether the expression of a translation-related factor is high may be determined using the expression of the translation-related factor in a cell from a normal subject or in a cell from a patient with a benign disease such as a benign tumor as a basis. Alternatively, whether the expression of a translation-related factor is high may also be determined using the expression amount of a housekeeping gene such as glyceraldehyde-3-phosphate dehydrogenase (GAPDH) or β-actin or of a product thereof as a basis.

In the sixth aspect, the present invention provides a kit for predicting a residence time of BSH, a complex comprising BSH, or a BSH derivative in a cancer cell, the kit comprising means for examining expression of a translation-related factor. The kit can be used to perform the method of the fifth aspect. The means for examining expression of CD44 is known to those skilled in the art and is not particularly limited, but preferred examples of the means include immunostaining with an antibody against a translation-related factor, and expression analysis of a translation-related factor gene using Northern blot analysis or real-time PCR. The kit may thus comprise an antibody against a translation-related factor. The kit may comprise a reagent or an instrument for performing immunostaining with an antibody against the translation-related factor. The kit may comprise a reagent or an instrument for Northern blot analysis or real-time PCR for examining expression of a translation-related factor. The kit typically comprises an instruction.

For other details of the method of the fifth aspect and the kit of the sixth aspect, the descriptions for the inventions according to the first to the fourth aspects of the present invention are applied.

In the seventh aspect, the present invention provides a method for predicting a sensitivity of a cancer cell to BNCT using BSH, a complex comprising BSH, or a BSH derivative, the method comprising examining expression of a translation-related factor in the cancer cell in a sample.

When the residence time in a cancer cell of BSH to which a peptide containing a basic amino acid residue is covalently linked is long, the intracellular concentration thereof is maintained high, thus the sensitivity of the cancer cell to BNCT is increased. Thus, it can be predicted that the higher the expression of a translation-related factor in a cancer cell, the higher the sensitivity of the cancer cell to BNCT using BSH, a complex comprising BSH, or a BSH derivative in the cancer cell.

In the eighth aspect, the present invention provides a kit for determining a sensitivity of a cancer to BNCT using BSH, a complex comprising BSH, or a BSH derivative, the kit comprising means for examining expression of a translation-related factor. The kit can be used to perform the method of the seventh aspect. The kit typically comprises an instruction.

According to the method of the seventh aspect and the kit of the eight aspect of the present invention, it is possible not only to know whether a cancer of a patient is a case suitable for BNCT using BSH, a complex comprising BSH, or a BSH derivative, but also to optimize the type of appropriate boron formulation in BNCT (for example, whether BSH, a complex comprising BSH, and/or a BSH derivative is effective, BPA is effective, or a combination thereof is effective) and to optimize the dosage or the mixed amount of the boron formulation.

For other details of the method of the seventh aspect and the kit of the eight aspect of the present invention, the descriptions for the method of the fifth aspect and the kit of the sixth aspect of the present invention are applied.

As mentioned above, the higher the expression of CD44, the higher the efficiency of introduction of a complex comprising a peptide containing a basic amino acid residue and BSH, or BSH to which a peptide containing a basic amino acid residue is covalently linked. Then, the higher the expression of CD44, the higher the sensitivity to BNCT using a complex comprising a peptide containing a basic amino acid residue and BSH, or BSH to which a peptide containing a basic amino acid residue is covalently linked. Thus, with the examination of the expression of CD44 in a cancer cell, it can be predicted that the higher the expression of CD44, the higher the possibility that BNCT is effective using a complex comprising a peptide containing a basic amino acid residue and BSH, or BSH to which a peptide containing a basic amino acid residue is covalently linked. Accordingly, in the ninth aspect, the present invention provides a method for predicting a possibility that BNCT is effective using a complex comprising a peptide containing a basic amino acid residue and BSH, or BSH to which a peptide containing a basic amino acid residue is covalently linked, the method comprising: examining an expression of CD44 in a cancer cell in a sample; and predicting that the higher the expression of CD44, the higher the possibility that BNCT is effective using a complex comprising a peptide containing a basic amino acid residue and BSH, or BSH to which a peptide containing a basic amino acid residue is covalently linked.

Furthermore, in the tenth aspect, the present invention provides a kit for predicting a possibility that BNCT is effective using a complex comprising a peptide containing a basic amino acid residue and BSH, or BSH to which a peptide containing a basic amino acid residue is covalently linked, the kit comprising means for examining expression of CD44. The kit can be used to perform the method of the ninth aspect. The kit typically comprises an instruction.

As mentioned above, the higher the expression of a translation-related factor in a cancer cell, the longer the residence time of BSH, a complex comprising BSH, or a BSH derivative. Then, the higher the expression of a translation-related factor in a cancer cell, the higher the sensitivity to BNCT using BSH, a complex comprising BSH, or a BSH derivative. Thus, with the examination of the expression of a translation-related factor in a cancer cell, it can be predicted that the higher the expression of the translation-related factor, the higher the possibility of success of BNCT using a complex comprising a peptide containing a basic amino acid residue and BSH, or BSH to which a peptide containing a basic amino acid residue is covalently linked. Accordingly, in the eleventh aspect, the present invention provides a method for predicting a possibility that BNCT is effective using BSH, a complex comprising BSH or a BSH derivative, the method comprising: examining an expression of a translation-related factor in a cancer cell in a sample; and predicting that the higher the expression of the translation-related factor, the higher the possibility that BNCT is effective using BSH, a complex comprising BSH or a BSH derivative.

Furthermore, in the twelfth aspect, the present invention provides a kit for predicting a possibility that BNCT is effective using BSH, a complex comprising BSH, or a BSH derivative, the kit comprising means for examining expression of a translation-related factor. The kit can be used to perform the method of the eleventh aspect. The kit typically comprises an instruction.

For the methods and kits according to the ninth to the twelfth aspects of the present invention, the descriptions for the methods and kits according to the first to eighth aspects are applied.

Since BPA is taken up into cells through amino acid transporters such as LAT1, LAT2, ATB^{0,+}, the uptake of BPA is higher as the expression of these amino acid transporters in a cancer cell is higher. Thus, for BPA in cancer cells, with the examination of the expression of an amino acid transporter such as LAT1, LAT2 or ATB^{0,+}, it can be predicted that the higher the expression of the amino acid transporter, the higher the possibility that BNCT is effective using BPA.

To summarize the above, the following can be said: the higher the expression of CD44 in a cancer cell, the more appropriate the selection of a boron formulation containing a complex comprising a peptide containing a basic amino acid residue and BSH, or BSH to which a peptide containing a basic amino acid residue is covalently linked. It can also be said that the higher the expression of an amino acid transporter such as LAT1, LAT2, ATB^{0,+}, (preferably LAT1) in a cancer cell, the more appropriate the selection of a boron formulation containing BPA. Accordingly, in the thirteenth aspect, the present invention provides a method for selecting a boron formulation for a boron neutron capture therapy (BNCT), comprising the steps of:
(a) examining expression of CD44 and expression of LAT1 in a cancer cell in a sample; and then
(b) selecting a boron formulation containing a complex comprising a peptide containing a basic amino acid residue and BSH, or BSH to which a peptide containing a basic amino acid residue is covalently linked when the expression of CD44 is higher than the expression of LAT1, and selecting a boron formulation containing BPA when the expression of LAT1 is higher than the expression of CD44.

Furthermore, in the fourteenth aspect, the present invention provides a kit for use in selecting a boron formulation for BNCT, the kit comprising means for examining expression of CD44 and means for examining expression of LAT1. The kit can be used to perform the method of the thirteenth aspect. The kit typically comprises an instruction.

It can also be said that the higher the expression of a translation-related factor in a cancer cell, the more appropriate the selection of a boron formulation containing BSH, a complex comprising BSH, and/or a BSH derivative. Furthermore, the higher the expression of an amino acid transporter such as LAT1, LAT2, ATB^{0,+} (preferably LAT1) in a cancer cell, the more appropriate the selection of a boron formulation containing BPA. Accordingly, in the fifteenth aspect, the present invention provides a method for selecting a boron formulation for a boron neutron capture therapy (BNCT), comprising the steps of:
(a) examining expression of a translation-related factor and expression of LAT1 in a cancer cell in a sample; and then
(b) selecting a boron formulation containing BSH, a complex comprising BSH, and/or a BSH derivative when the expression of a translation-related factor is higher than the expression of LAT1, and selecting a boron formulation containing BPA when the expression of LAT1 is higher than the expression of a translation-related factor.

Furthermore, in the sixteenth aspect, the present invention provides a kit for use in selecting a boron formulation for BNCT, the kit comprising means for examining expression of a translation-related factor and means for examining expression of LAT1. The kit can be used to perform the method of the fifteenth aspect. The kit typically comprises an instruction.

In the methods and kits of the present invention according to the thirteenth to the sixteenth aspects, the expression amounts of CD44, a translation-related factor, and LAT in a cancer cell are examined. The expression amounts of CD44 and a translation-related factor are examined as described above. As used herein, LAT1 means either one or both of a LAT1 gene and a LAT1 protein. Expression of LAT1 can be examined by known methods. For example, expression of LAT1 can be examined by detecting a LAT1 protein in a cancer cell by immunostaining with an anti-LAT1 antibody, or be examining expression of a LAT1 gene in a cancer cell by Northern blot analysis or real-time PCR. Means and methods for examining expression of LAT1 in a cancer cell are not limited thereto.

Examples of preferred methods for examining expression of LAT1 include immunostaining with an anti-LAT1 antibody. Anti-LAT1 antibodies can be obtained by known methods and are also commercially available. The anti-LAT1 antibody may be a polyclonal antibody or a monoclonal antibody, and preferably, a monoclonal antibody. Immunostaining is a know method. It is preferable to use an anti-LAT1 antibody to which a detectable label such as a fluorescent dye, a fluorescent protein, or a radioisotope is attached, for immunostaining. Secondary antibodies can also be used to detect the anti-LAT1 antibody. The anti-LAT1 antibody may be an antibody against a LAT1 protein or a portion thereof, or may be an antibody against a gene encoding a LAT1 protein or a portion thereof.

Determination of whether LAT1 expression is high can also be performed by known methods. For example, whether the expression of LAT1 is high may be determined using the expression of LAT1 in a cell from a normal subject, or in a cell from a patient with a benign disease such as a benign tumor as a basis. Alternatively, whether the expression of LAT1 is high may be determined using the expression amount of a housekeeping gene such as glyceraldehyde-3-phosphate dehydrogenase (GAPDH) or β-actin or of a product thereof as a basis.

In addition to or in place of expression of LAT1, expression of an amino acid transporter specific for tyrosine or phenylalanine, such as LAT 2 and/or ATB^{0,+}, may be examined.

Means and methods for comparing expression of CD44 to expression of LAT1 are known to those skilled in the art. One example thereof is to use antibodies against these proteins, in which labels identifiable to each other are attached to the antibodies. For example, the antibody against CD44 may be labeled with a green fluorescent protein and the antibody against LAT1 may be labeled with a red fluorescent protein, and the expression of the two may be visually compared. Another example thereof is to compare the difference between expression of CD44 in a cancer cell and in a normal cell with the difference between expression of LAT1 in a cancer cell and in a normal cell, e.g., using a statistical method. A comparison of expression of a translation-related factor with expression of LAT1 can be performed in the same manner.

In the methods and kits according to the thirteenth to sixteenth aspects, when the expression of CD44 is higher than the expression of LAT1, a boron formulation containing a complex comprising a peptide containing a basic amino acid residue and BSH, or BSH to which a peptide containing a basic amino acid residue is covalently linked is selected. When the expression of a translation-related factor is higher than the expression of LAT1, a boron formulation including BSH, a complex comprising BSH, and/or a BSH derivative is selected. When the expression of LAT1 is higher than the expression of CD44, or when the expression of LAT1 is higher than the expression of a translation-related factor, a boron formulation containing BPA is selected. When BNCT is performed using the boron formulation selected by the methods and kits of these aspects, the selected formulation may be used alone, or may be used in combination or formulated with another boron formulation. For example, when BPA is selected, BPA may be used alone, or in combination of BPA and a complex comprising BSH and BPA may be used. The dosage of boron formulation, the ratio thereof to another boron formulation, and the like can be readily determined by a physician.

For other details of the methods and the kits according to the thirteenth to the sixteenth aspects of the present invention, the descriptions for the methods and the kits according to the first to the twelfth aspects are applied.

The meaning of the terms used herein is the same as commonly understood in the art such as medicine, pharmacy, biology, unless otherwise specified.

Hereinafter, the present invention is described in more detail and specifically with Examples, but Examples should not be construed as any limitation of the scope of the present invention.

### EXAMPLES

### Example 1

Example 1. Uptake of BSH to which a Peptide Containing a Basic Amino Acid Residue is Covalently Linked and a Complex Comprising a Peptide Containing a Basic Amino Acid Residue and BSH, into a CD44-Expressing Cancer Cell and a CD44-Knocked Down Cancer Cell

To examine the relationship between intracellular uptake of BSH to which a peptide consisting of three arginine is covalently linked (referred to as BSH-3R), BSH to which a peptide consisting of eleven arginine is covalently linked (referred to as BSH-11R), and a complex comprising Ala-Ala-Ala-Ala-Ala-Ala-Lys and BS (referred to BSH/A6K) and CD44 expression, a CD44-shRNA lentivirus was first introduced into a malignant brain tumor cell line U87MG, which was highly expressing CD44, to produce a CD44-knocked down cell subline. The knockdown efficiency was confirmed by Western blotting method with an anti-CD44 antibody (manufactured by Cell Signaling Technology, Inc.). Then, to verify the correlation of the introduction efficiency of BSH-11R and BSH/A6K with the expression level of CD44, the intracellular uptake of BSH-11R and BSH/A6K into a CD44-knocked down cell subline produced by the method described above was examined by performing fluorescent immunostaining with an antibody against BSH. The antibody used was a mouse monoclonal anti-BSH antibody (provided by Dr. Kirihata, Osaka Prefecture University). BSH-3R and BSH-11R were obtained by chemical synthesis. BSH/A6K was obtained by the method described in International Patent Application Publication WO 2018/097335 A1, which is incorporated herein by reference.

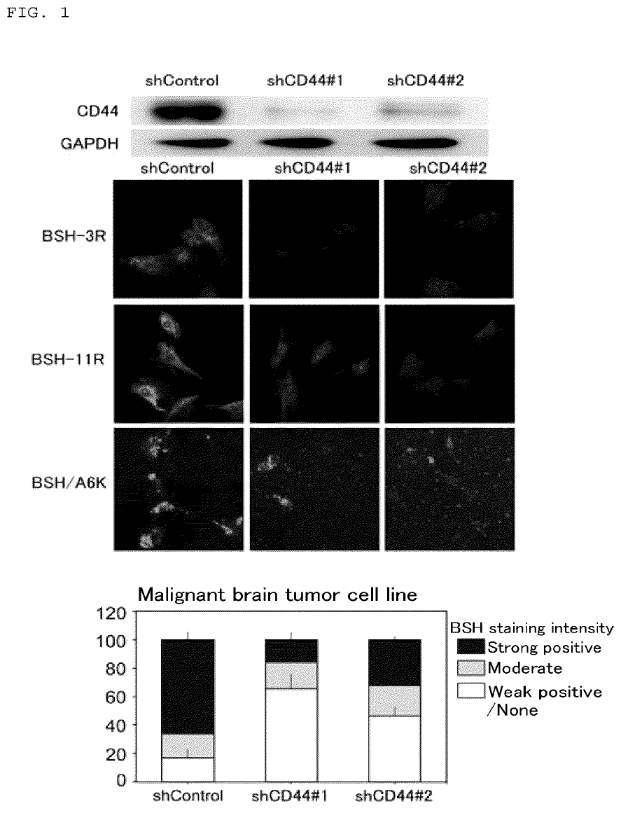

The results are shown in FIG. 1. As can be seen from western blotting shown in the upper section of FIG. 1, CD44 was definitely knocked down in the produced two cell sublines. As shown in the middle section of FIG. 1, it was confirmed that the lower the expression of CD44, the more reduction of the intracellular uptakes of BSH-3R, BSH-11R, and BSH/A6K (the lower section of FIG. 1 is a bar graph of the result when BSH-11R was used). In other words, it was confirmed that the higher the expression of CD44, the more increase the intracellular uptake of BSH-3R, BSH-11R, and BSH/A6K has.

### Example 2

### Example 2. Targeting of Translation-Related Factors by BSH to which a Peptide Containing a Basic Amino Acid Residue is Covalently Linked

To a cell crushing solution of a malignant brain tumor cell line U251, simple BSH (referred to BSH) (50 μM) or BSH-11R (50 μM) was added, and proteins which bind to BSH and BSH-11R were explored by immunoprecipitation with an anti-BSH antibody (10 μg).

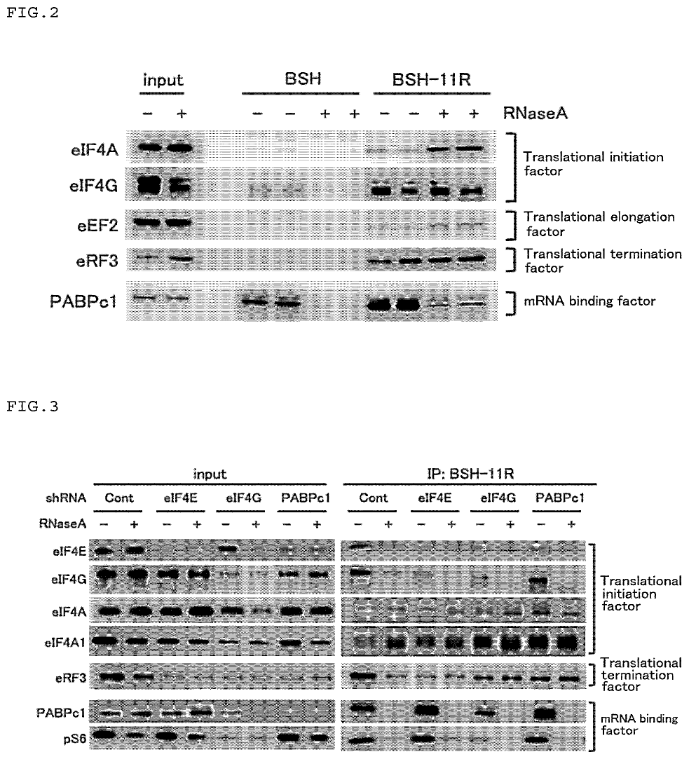

The results are shown in FIG. 2. Since more translation-related factors were co-precipitated with BSH-11R compared to BSH, it was found that the peptide portion (11R) of BSH-11R is necessary for binding to translation-related factors. It was also found that there are mRNA-independent and mRNA-dependent binding patterns according to the translation-related factors.

To examine whether the binding of BSH-11R to the translation-related factors is a direct or indirect binding, a subline of malignant brain tumor cell line U251 that underwent knockdown of the translation-related factors with shRNA lentivirus was prepared. To the cell crushing solutions, BSH-11R (50 μM) was added, and translation-related factors that bind to BSH-11R were analyzed with an anti-BSH antibody (10 μg).

The results are shown in FIG. 3. It was found that BSH-11R binds directly (without RNA) to eIF4A and eRF3, and eIF4E and eIF4G bind indirectly to BSH-11R via eIF4A. It was also concluded, as combined with the results in FIG. 2, that PABPc1 binds to BSH in an RNA-dependent manner.

### Example 3

### Example 3. BNCT Using BSH to which a Peptide Containing a Basic Amino Acid Residue is Covalently Linked

From the binding of BSH-11R to translation-related factors, it was thought that the translation mechanism could be selectively targeted. It was thus decided to perform BNCT using BSH-11R. Malignant brain tumor cell lines U87ΔEGFR were pretreated with 100 μM BSH-11R (24 hours prior to irradiation). The cells were stripped with trypsin on the day of irradiation, dispensed into tubes at an amount of 1×10⁶ cells/mL, and irradiated with neutron rays using an accelerator (at the National Institute of Radiological Sciences). The irradiation times were 0, 15, 45 and 60 minutes, and every substantial Gy (gray) was estimated and calculated to be less than or equal to 1 Gy.

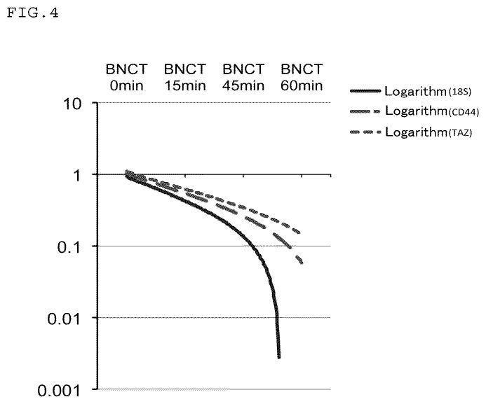

The cells after irradiation were recovered, RNA extraction was performed, and then cDNA was produced using an equal amount of RNA (1000 ng) as a template, and quantitative PCR was performed. As the result, it was found as shown in FIG. 4 that the proportion of 18S ribosomal RNA and mRNA decreased significantly depending on the irradiation time with neutron rays. No such changes were observed in high-dose irradiation (10 Gy) with x-rays performed as a control experiment. From these, it could be determined that BNCT using BSH-11R was able to destroy the translation mechanism extremely efficiently.

### Example 4

### Example 4. Comparison of Expression Levels of LAT1 and CD44 in Various Carcinomas

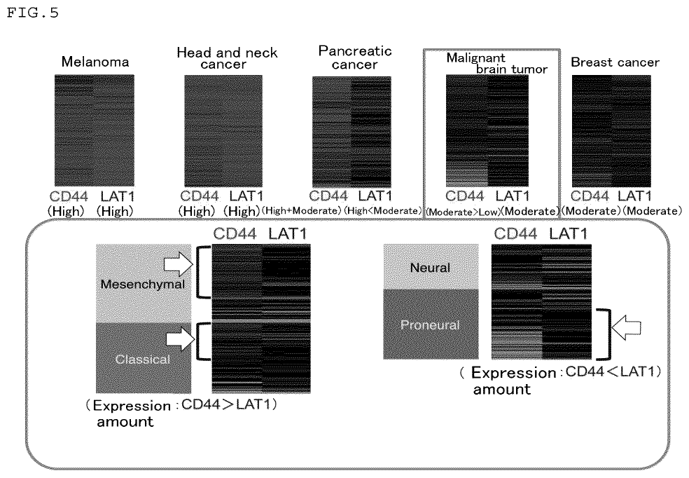

FIG. 5 shows the results of examining the expression intensities of CD44 and LAT1 for specimens of melanoma, head and neck cancer, pancreatic cancer, malignant brain tumor, and breast cancer (clinical specimens registered in the TCGA database) by an immunostaining method.

In melanoma and head and neck cancer, there were many cases in which both CD44 and LAT1 were highly expressed. From these results, it was considered that BNCT for melanoma and head and neck cancers responds in many cases when any one of a complex comprising a peptide containing a basic amino acid residue and BSH, BSH to which a peptide containing a basic amino acid residue is covalently linked, or BPA is selected. It was also considered that BNCT for melanoma and head and neck cancers is effective in many cases when a combination of a complex comprising a peptide containing a basic amino acid residue and BSH or BSH to which a peptide containing a basic amino acid residue is covalently linked with BPA is selected.

In pancreatic cancer, there were many cases in which CD44 was highly or moderately expressed. For LAT1, there were more cases with moderate expression than those with high expression. From these results, it was considered that BNCT for pancreatic cancer responds in many cases when a complex comprising a peptide containing a basic amino acid reside and BSH, or BSH to which a peptide containing a basic amino acid residue is covalently linked is selected, and that it responds to a certain degree in many cases when BPA is selected. It was also considered, in a combination use of a complex comprising a peptide containing a basic amino acid residue and BSH or a peptide containing a basic amino acid residue with BPA, that BNCT for pancreatic cancer is effective in many cases when the ratio of the complex comprising a peptide containing a basic amino acid residue and BSH or the peptide containing a basic amino acid residue is high.

In breast cancer, there were many cases in which both CD44 and LAT1 were moderately expressed. From these results, it was considered that BNCT for breast cancer is expected to respond to a certain degree in many cases when any one of the complex comprising a peptide containing a basic amino acid residue and BSH, BSH to which a peptide containing a basic amino acid residue is covalently linked, or BPA is selected. It was also considered that BNCT for breast cancer is expected to respond to a certain degree in many cases when a combination of a complex comprising a peptide containing a basic amino acid residue and BSH or BSH to which a peptide containing a basic amino acid reside is covalently linked with BPA is used.

In malignant brain tumors, for CD44, there were many cases with moderate expression, and also quite many cases with low expression (there were few cases with high expression). For LAT1, there were many cases with moderate expression. From these results, it was considered that BNCT for malignant brain tumors is expected to be effective to a certain degree in many cases when BPA is selected. It was also considered that BNCT for malignant brain tumors is expected to respond in fewer cases when a complex comprising a peptide containing a basic amino acid residue and BSH or BSH to which a peptide containing a basic amino acid residue is covalently linked is selected than when BPA is selected. Furthermore, it was considered, in a combination use of a complex comprising a peptide containing a basic amino acid residue and BSH or a peptide containing a basic amino acid residue with BPA, that BNCT for malignant brain tumors is effective in many cases when a ratio of BPA contained is high.

Malignant brain tumors were studied by type. The results are shown in the lower column of FIG. 5. In the mesenchymal and classical brain tumors, there are many cases in which CD44 is more highly expressed than LAT1 (indicated by arrows in the figure). It was thus considered that BNCT for them is effective in many cases when a complex comprising a peptide containing a basic amino acid residue and BSH, or BSH to which a peptide containing a basic amino acid residue is covalently linked is selected. It was also considered, in a combination use of a complex comprising a peptide containing a basic amino acid residue and BSH or a peptide containing a basic amino acid residue with BPA, BNCT for them is effective in many cases when the ratio of the complex comprising a peptide containing a basic amino acid residue and BSH or the peptide containing a basic amino acid residue is high. In neural brain tumors, there were many cases in which both CD44 and LAT1 were moderately expressed. In anterior neural brain tumors, there were many cases in which LAT1 is more highly expressed than CD44 (indicated by arrows in the figure), thus it was considered that BNCT for them is effective in many cases when BPA is selected. It was also considered, in a combination use of a complex comprising a peptide containing a basic amino acid residue and BSH or BSH to which a peptide containing a basic amino acid residue is covalently linked with BPA, that BNCT for them is effective in many cases when a ratio of BPA contained is high.

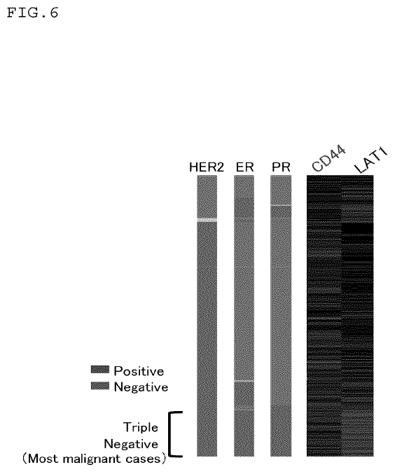

Breast cancer was also studied by type. The results are shown in FIG. 6. In the most malignant cases where all of the HER2, estrogen and progesterone receptors are negative, there are many cases in which LAT1 is more highly expressed than CD44. It was thus considered that BNCT for them is effective in many cases when BPA is selected. It was also considered, in a combination use of a complex comprising a peptide containing a basic amino acid residue and BSH or BSH to which a peptide containing a basic amino acid residue is covalently linked with BPA, BNCT for them is effective in many cases when a ratio of BPA compounded is high.

General CD44 and LAT1 expression may be examined for each carcinoma, and expression of CD44 and LAT1 may be analyzed for each case. Personalized medicine of BNCT can be achieved by case-by-case analysis.

From the above results, it can be said that, with the methods and kits of the present invention, the optimal selection and determination of the combination or ratio of boron formulations can be performed for each carcinoma/case, and the potential of BNCT can be expanded for carcinoma/cases that have not previously been considered a good indication for BNCT using BPA.

This application claims the benefit of priority to Japanese Patent Application No. 2018-025216 filed on Feb. 15, 2018. The entire contents of the Japanese application are incorporated herein by reference. Furthermore, the entire contents of the literatures cited herein are also incorporated herein by reference.

### INDUSTRIAL APPLICABILITY

The present invention can be used in testing and research of cancer, research, development, and manufacture of anticancer agents, and the like.

1-20. (canceled)

21. A method of cancer treatment in a subject in need thereof using boron neutron capture therapy (BNCT), the method comprising: 1) measuring expression of CD44 in a cancer cell in a sample from the subject relative to control cells; 2) if the expression of CD44 measured in the cancer cell is high, administering a complex comprising mercaptoundecahydrodecaborate (BSH)/A6K (SEQ ID NO: 4), or BSH covalently linked to a peptide comprising RRR (BSH-3R) or RRRRRRRRRRR (BSH-11R; SEQ ID NO:1); 3) waiting for selective uptake of the BSH composition by the subject's cancer cells; and 4) subjecting the subject to BNCT, thereby treating the subject.

22. (canceled)

23. A method of cancer treatment in a subject in need thereof using BNCT, the method comprising: 1) measuring expression of a translation-related factor in a cancer cell in a sample from the subject relative to control cells; 2) if the expression of the translation-related factor measured in the cancer cell is high, administering a composition comprising BSH/A6K (SEQ ID NO:4), or BSH covalently linked to a peptide comprising RRR (BSH-3R) or RRRRRRRRRRR (BSH-11R; SEQ ID NO:1); 3) waiting for selective uptake of the BSH composition by the subject's cancer cells; and 4) subjecting the subject to BNCT, thereby treating the subject.

24. (canceled)

25. A method of cancer treatment in a subject in need thereof using boron neutron capture therapy (BNCT), the method comprising the steps of:
(a) examining expression of CD44 and expression of LAT1 in a cancer cell in a sample relative to controls; and then
(b) selecting a boron formulation containing BSH from the group consisting of BSH/A6K (SEQ ID NO:4), or BSH covalently linked to a peptide comprising RRR (BSH-3R) or RRRRRRRRRRR (BSH-11R; SEQ ID NO:1) when the expression of CD44 is higher than the expression of LAT1, or selecting a boron formulation containing BPA when the expression of LAT1 is higher than the expression of CD44;
(c) administering the selected formulation to the subject; and
(d) subjecting the subject to BNCT, thereby treating the subject.

26. (canceled)

27. A method of cancer treatment in a subject in need thereof by selecting a boron formulation for boron neutron capture therapy (BNCT), comprising the steps of:
(a) examining expression of a translation-related factor and expression of LAT1 in a cancer cell in a sample from the subject;
(b) selecting a boron formulation containing BSH when the expression of a translation-related factor is higher than the expression of LAT1, or selecting a boron formulation containing BPA when the expression of LAT1 is higher than the expression of a translation-related factor
(c) administering the selected compound to the subject;
(d) treating the subject with BNCT, thereby treating the subject's cancer.

28. (canceled)

29. A method for treating a cancer of a subject with boron neutron capture therapy (BNCT), the method comprising:
administering to a subject in need thereof a complex comprising A6K and mercaptoundecahydrodecaborate (BSH), or BSH-3R, or BSH-11R, or a combination thereof,
wherein the cancer highly expresses CD44.

30. A method for treating a subject's cancer with boron neutron capture therapy (BNCT), the method comprising:
administering to a subject in need thereof a complex comprising BSH/A6K, BSH-3R or BSH-11R,
wherein the cancer highly expresses a translation-related factor.

31. The method according to claim 30, wherein the translation-related factor is one or more selected from the group consisting of eIF4A, eIF4E, eIF4G, eEF2, eRF3, pS6, and PABPc1.

32. The method according to claim 30, wherein the expression of a translation-related factor is examined by immunostaining.
